(19) United States
(12) Patent Application Publication
(10) Pub. No.: US 2024/0228413 A1
(43) Pub. Date: Jul. 11, 2024

(54) METHOD OF PREPARING ALIPHATIC POLYOLS HAVING THREE OR MORE HYDROXY GROUPS AND A TOTAL NUMBER OF CARBON ATOMS OF 5 OR MORE

(71) Applicant: BASF SE, Ludwigshafen am Rhein (DE)

(72) Inventors: Sylvester GROESSL, Ludwigshafen (DE); Stephanie Sybille LINKE, Ludwigshafen (DE); Grit BAIER, Ludwigshafen (DE); Tavares Andre Rute da Conceicao, Ludwigshafen (DE)

(21) Appl. No.: 18/555,120

(22) PCT Filed: May 4, 2022
(86) PCT No.: PCT/EP22/61908

§ 371 Date: Oct. 12, 2023

(30) Foreign Application Priority Data
May 4, 2021 (EP) 21172092.5

(51) Int. Cl. C07C 29/149

(52) U.S. Cl. CPC C07C 29/149

(57) ABSTRACT

Disclosed herein are a method of preparing a product including one or more product compounds selected from the group consisting of aliphatic polyols having three or more hydroxy groups and a total number of carbon atoms of 5 or more, a method of using compounds selected from the group consisting of aliphatic tricarboxylic acids, esters thereof, anhydrides thereof, and salts thereof as starting compounds for making the product compounds, and a method of using a heterogeneous hydrogenation catalyst in a method of preparing a product including one or more of the product compounds.

The present application relates to a method of preparing a product comprising (a) one or more product compounds selected from the group consisting of aliphatic polyols having three or more hydroxy groups and a total number of carbon atoms of 5 or more, and/or (b) one or more reaction products of one or more of said compounds selected from the group consisting of aliphatic polyols having three or more hydroxy groups and a total number of carbon atoms of 5 or more, to such products (a) and (b), and to the use of compounds selected from the group consisting of aliphatic tricarboxylic acids, esters thereof, anhydrides thereof and salts thereof as starting compounds for making product compounds selected from the group consisting of aliphatic polyols having three or more hydroxy groups and a total number of carbon atoms of 5 or more. Also described is the use of a heterogeneous hydrogenation catalyst in a method of preparing a product comprising one or more product compounds selected from the group consisting of aliphatic polyols having three or more hydroxy groups and a total number of carbon atoms of 5 or more.

Radial polyols having a core structure comparable to 1,1,1-trimethylolpropane (TMP) or pentaerythritol (PETP) are an important synthetic target, because of their broad application scope on an industrial scale. Presently, polyols (such as TMP, di-TMP, PETP, propanediol, pentanediol, etc.) are produced mainly petrochemically. Due to the finite nature and instability of fossil feedstock supply and for environmental reasons, replacement of fossil feedstock by non-fossil feedstock, i.e. feedstock obtained from renewable resources, becomes more and more important.

Radial tricarboxylic acids, e.g. citric acid, isocitric acid, tricarballylic acid and aconitic acid, esters thereof, anhydrides thereof, and salts thereof, could be used as precursors for radial polyols having three hydroxy groups, provided that selective hydrogenation of the carboxylic groups to hydroxy groups is achieved. Tricarboxylic acids like citric acid and aconitic acid are obtainable from renewable resources, e.g. from plant material by sugar fermentation.

Unfortunately, tricarboxylic acids are difficult substrates for typical reduction procedures due to the following major reasons:
a pKA value which is incompatible with a lot of catalyst materials
a low electrophilicity of the central C═O bond
a strong tendency to decarboxylate with increased temperature
a low solubility in most organic solvents
a high reduction potential (which for example leads to neutralization with sodium hydride, but to reduction only with stronger reducing agents like lithium aluminum hydride).

Citric acid in particular is very prone to decarboxylation and dehydration at an increased temperature.

DE 42 33 431 A1 discloses a process for the preparation of propane-1,2,3-tricarboxylic acid, tetrahydrofurfurylacetic acid and the C₁- to C₂₀-alkyl or C₇- to C₁₂-aralkyl esters thereof, propane-1,2,3-trimethanol, 3-methyltetrahydrofuran, 3-(2′-hydroxyethyl)tetrahydrofuran, 4-hydroxymethyltetrahydropyran, 2-methyl-gamma-butyrolactone and/or 3-methyl-gamma-butyrolactone. Said process comprises reacting citric acid or the C₁- to C₂₀-alkyl or C₇- to C₁₂-aralkyl esters thereof on hydrogenation catalysts in non-aqueous solvents at 50° ° C. to 400° C. and 1 bar to 400 bar. For the hydrogenation to propane-1,2,3-trimethanol, low reaction temperatures, high reaction pressures and short residence times are favorable. Propane-1,2,3-trimethanol is preferentially formed at from 100° C. to 250° C., in particular at from 125° C. to 175° C., and at from 100 bar to 400 bar, in particular at from 150 bar to 300 bar. In example 7, 400 ml of triethyl citrate were hydrogenated at 150° C. and 200 bar in 1100 ml of tetrahydrofuran using 60 g of a catalyst comprising 37 wt. % CuO, 1 wt. % BaO, 1 wt. % Cr₂O3, 0.4 wt.-% ZnO, 15 wt.-% MgO, 29 wt.-% SiO₂ (“catalyst D”) until the take-up of hydrogen had ceased. The reaction product was freed from tetrahydrofuran and distilled under reduced pressure, giving 87 g (39%) of propane-1,2,3-trimethanol and 41.6 g (19%) of 3-(2′-hydroxyethyl)tetrahydrofuran.

US 2018/0346619 A1 mentions a process wherein aconitic acid is reduced by lithium aluminum hydride (LiAlH₄) in diethylether, producing 3-(hydroxymethyl)-2-pentene-1,5-diol which in a further process is reduced in ethanolic solution with hydrogen gas and a palladium on carbon (Pd/C) catalyst, producing propane-1,2,3-trimethanol.

Related art is also
U.S. Pat. No. 5,731,479 AUS 2018/0346688 A1.

It is a primary object of the present invention to provide a method for preparing a product comprising (a) one or more product compounds selected from the group consisting of aliphatic polyols having three or more hydroxy groups and a total number of carbon atoms of 5 or more and/or (b) one or more reaction products of one or more of said compounds selected from the group consisting of aliphatic polyols having three or more hydroxy groups and a total number of carbon atoms of 5 or more, wherein starting material obtained from renewable sources may be used.

It is a further object to provide a method for selective hydrogenation of carboxyl groups, carboalkoxy groups, anhydride groups and/or carboxylate groups of aliphatic tricarboxylic acids, esters thereof, anhydrides thereof, and salts thereof, so that aliphatic polyols having three or more hydroxy groups and a total number of carbon atoms of 5 or more are obtained in a useful yield, and undesired side reactions which may result inter alia in loss of two or more carbon atoms by decarboxylation, incomplete hydrogenation and ring-closing are suppressed.

The primary object and other objects of the present invention can be accomplished by a method of preparing a product comprising
(a) one or more product compounds selected from the group consisting of aliphatic polyols having three or more hydroxy groups and a total number of carbon atoms of 5 or more
and/or
(b) one or more reaction products of one or more of said compounds selected from the group consisting of aliphatic polyols having three or more hydroxy groups and a total number of carbon atoms of 5 or more,
comprising at least the following steps:
(i) providing or preparing a starting material comprising one or more starting compounds selected from the group consisting of aliphatic tricarboxylic acids, esters thereof, anhydrides thereof, and salts thereof,
(ii) providing or preparing a solvent having a dielectric constant above the dielectric constant of n-butanol,
and
(iii) chemically converting said one or more starting compounds provided or prepared in step (i)
   at a temperature in the range of from 80° C. to 155° C.,
   at a partial pressure of hydrogen in the range of from 15 MPa to 30 MPa,
   in said solvent provided or prepared in step (ii), and
   in the presence of a heterogeneous hydrogenation catalyst comprising one or more metals selected from the group consisting of Mn, Re, Fe, Ru, and Os,
so that carboxyl groups, carboalkoxy groups, anhydride groups and/or carboxylate groups as present in the one or more starting compounds are selectively hydrogenated to the corresponding hydroxy groups so that a product results comprising one or more product compounds selected from the group consisting of aliphatic polyols having three or more hydroxy groups and a total number of carbon atoms of 5 or more.

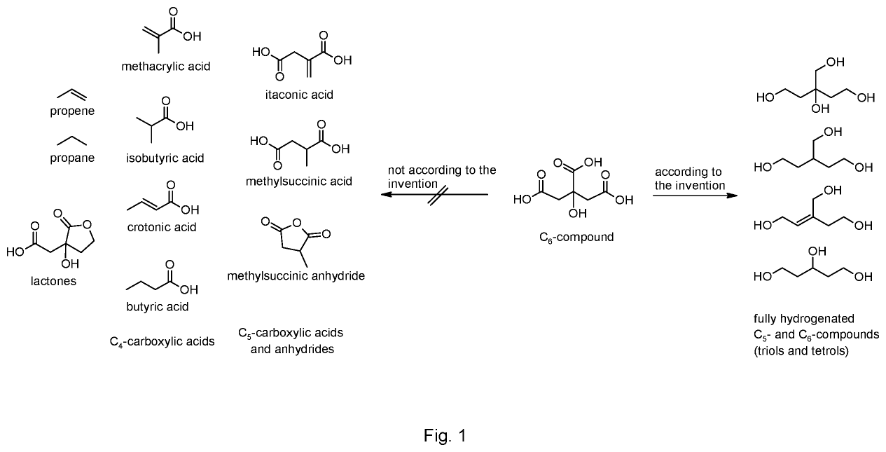

Surprisingly it has been found that selective hydrogenation of carboxyl groups, carboalkoxy groups, anhydride groups, resp. carboxylate groups of aliphatic tricarboxylic acids, esters thereof, anhydrides thereof, and salts thereof is achieved and product compounds selected from the group consisting of aliphatic polyols having three or more hydroxy groups and a total number of carbon atoms of 5 or more may be obtained in a useful yield, when the parameters of the hydrogenation reaction in step (iii) are selected as defined above, and a heterogeneous hydrogenation catalyst as defined above and a solvent as defined above are used. Thus, when the chemical conversion of the starting compound(s) in step (iii) is carried out using the parameters as defined above, a heterogeneous hydrogenation catalyst as defined above and a solvent as defined above, undesirable side reactions are suppressed (cf. FIG. 1 which for the starting compound citric acid shows formation of aliphatic polyols having three or more hydroxy groups and a total number of carbon atoms of 5 or more, and products of possible side reactions).

In above-defined case (a) the product resulting in step (iii), which comprises one or more product compounds selected from the group consisting of aliphatic polyols having three or more hydroxy groups and a total number of carbon atoms of 5 or more, is the final product of the method. Herein, the final product comprises or consists of one, two or more product compounds selected from the group consisting of aliphatic polyols having three or more hydroxy groups and a total number of carbon atoms of 5 or more.

In above-defined case (b) the product resulting in step (iii), which comprises one or more product compounds selected from the group consisting of aliphatic polyols having three or more hydroxy groups and a total number of carbon atoms of 5 or more, is an intermediate product, and said intermediate product is transferred into a final product comprising one or more reaction products of one or more of said compounds selected from the group consisting of aliphatic polyols having three or more hydroxy groups and a total number of carbon atoms of 5 or more by conducting one or more additional steps following above-defined step (iii). Herein, the intermediate product comprises or consists of one, two or more product compounds selected from the group consisting of aliphatic polyols having three or more hydroxy groups and a total number of carbon atoms of 5 or more, and the final product comprises or consists of one, two or more reaction products of one, two or more of said compounds selected from the group consisting of aliphatic polyols having three or more hydroxy groups and a total number of carbon atoms of 5 or more (for details see below).

In step (i) of the above-defined method, a starting material comprising one or more starting compounds selected from the group consisting of
aliphatic tricarboxylic acids,
esters of aliphatic tricarboxylic acids anhydrides of aliphatic tricarboxylic acids
and salts of aliphatic tricarboxylic acids
   is prepared or provided. Among these starting compounds, aliphatic tricarboxylic acids are preferred.

When the starting compound is an aliphatic tricarboxylic acid, carboxyl groups are selectively hydrogenated to hydroxy groups. When the starting compound is a salt of an aliphatic tricarboxylic acid, carboxylate groups are selectively hydrogenated to hydroxy groups. When the starting compound is an ester of an aliphatic tricarboxylic acid, carboalkoxy groups are selectively hydrogenated to hydroxy groups. When the starting compound is an anhydride of an aliphatic tricarboxylic acid, anhydride group and possibly the remaining carboxyl group are selectively hydrogenated to hydroxy groups.

For instance, the starting material comprises or consists of one, two or more starting compounds selected from the group consisting of aliphatic tricarboxylic acids, esters thereof, anhydrides thereof, and salts thereof.

The starting compound or at least one of said starting compounds may be selected from the group consisting of
aliphatic tricarboxylic acids having a total number of carbon atoms of 6
esters of aliphatic tricarboxylic acids having a total number of carbon atoms of 6
anhydrides of aliphatic tricarboxylic acids having a total number of carbon atoms of 6
and salts of aliphatic tricarboxylic acids having a total number of carbon atoms of 6.

Among these starting compounds, aliphatic tricarboxylic acids having a total number of carbon atoms of 6 are preferred.

For instance, the starting material comprises or consists of one, two or more starting compounds selected from the group consisting of aliphatic tricarboxylic acids having a total number of carbon atoms of 6, esters thereof, anhydrides thereof, and salts thereof. Preferably, all of the starting compounds are selected from the group consisting of aliphatic tricarboxylic acids having a total number of carbon atoms of 6, esters thereof, anhydrides thereof, and salts thereof.

In certain cases, the starting compound or at least one of said starting compounds is selected from the group consisting of
tricarballylic acid,
esters of tricarballylic acid,
anhydrides of tricarballylic acid,
and salts of tricarballylic acid.

Among these starting compounds, tricarballylic acid is preferred.

For instance, the starting material comprises or consists of one, two or more starting compounds selected from the group consisting of tricarballylic acid, esters thereof, anhydrides thereof, and salts thereof. Preferably, all of said starting compounds are selected from the group consisting of tricarballylic acid, esters thereof, anhydrides thereof, and salts thereof.

In certain cases, the starting compound or at least one of said starting compounds is selected from the group consisting of
alpha-beta-unsaturated aliphatic tricarboxylic acids
esters of alpha-beta-unsaturated aliphatic tricarboxylic acids
anhydrides of alpha-beta-unsaturated aliphatic tricarboxylic acids
and salts alpha-beta-unsaturated aliphatic tricarboxylic acids.

Among these starting compounds, alpha-beta-unsaturated aliphatic tricarboxylic acids are preferred.

For instance, the starting material comprises or consists of one, two or more starting compounds selected from the group consisting of alpha-beta-unsaturated aliphatic tricarboxylic acids, esters thereof, anhydrides thereof and salts thereof. Preferably, all of said starting compounds are selected from the group consisting of alpha-beta-unsaturated aliphatic tricarboxylic acids, esters thereof, anhydrides thereof and salts thereof.

Preferably, the alpha-beta-unsaturated aliphatic tricarboxylic acid is aconitic acid. In this case, the starting compound or at least one of said starting compounds is selected from the group consisting of
aconitic acid,
esters of aconitic acid
anhydrides of aconitic acid,
and salts of aconitic acid.

Among these starting compounds, aconitic acid is preferred.

For instance, the starting material comprises or consists of one, two or more starting compounds selected from the group consisting of aconitic acid, esters thereof, anhydrides thereof, and salts thereof. Preferably, all of said starting compounds are selected from the group consisting of aconitic acid, esters thereof, anhydrides thereof, and salts thereof.

In certain cases, the starting compound or at least one of said starting compounds is selected from the group consisting of
alpha-functionalized aliphatic tricarboxylic acids
esters of alpha-functionalized aliphatic tricarboxylic acids
anhydrides of alpha-functionalized aliphatic tricarboxylic acids
and salts of alpha-functionalized aliphatic tricarboxylic acids.

Among these starting compounds, alpha-functionalized aliphatic tricarboxylic acids are preferred.

For instance, the starting material comprises or consists of one, two or more starting compounds selected from the group consisting of alpha-functionalized aliphatic tricarboxylic acids, esters thereof, anhydrides thereof and salts thereof. Preferably, all of said starting compounds are selected from the group consisting of alpha-functionalized aliphatic tricarboxylic acids, esters thereof, anhydrides thereof and salts thereof.

Preferably, the alpha-functionalized aliphatic tricarboxylic acids are alpha-hydroxy aliphatic carboxylic acids. In this case, the starting compound or at least one of said starting compounds is selected from the group consisting of
alpha-hydroxy aliphatic tricarboxylic acids,
esters of alpha-hydroxy aliphatic tricarboxylic acids
anhydrides of alpha-hydroxy aliphatic tricarboxylic acids
and salts of alpha-hydroxy aliphatic tricarboxylic acids.

Among these starting compounds, alpha-hydroxy aliphatic tricarboxylic acids are preferred.

For instance, the starting material comprises or consists of one, two or more starting compounds selected from the group consisting of alpha-hydroxy aliphatic tricarboxylic acids, esters thereof, anhydrides thereof and salts thereof. Preferably, all of said starting compounds are selected from the group consisting of alpha-hydroxy aliphatic tricarboxylic acids, esters thereof, anhydrides thereof and salts thereof.

Preferred alpha-hydroxy aliphatic tricarboxylic acids are citric acid and isocitric acid. In this case, the starting compound or at least one of said starting compounds is selected from the group consisting of
citric acid and isocitric acid,
esters of citric acid and esters of isocitric acid
anhydrides of citric acid and anhydrides of isocitric acid
salts of citric acid and salts of isocitric acid.

Among these starting compounds, citric acid and isocitric acid are preferred.

For instance, the starting material comprises or consists of one, two or more starting compounds selected from the group consisting of citric acid, isocitric acid, esters thereof, anhydrides thereof and salts thereof. Preferably, all of said starting compounds are selected from the group consisting of citric acid, isocitric acid, esters thereof, anhydrides thereof and salts thereof.

The most preferred starting compounds in the group consisting of alpha-hydroxy aliphatic tricarboxylic acids, esters thereof, anhydrides thereof and salts thereof are citric acid, triethyl citrate, and isocitric acid. For instance, the starting material comprises or consists of one, two or all of citric acid, triethyl citrate, and isocitric acid. Preferably, all of said starting compounds are selected from the group consisting of citric acid, triethyl citrate, and isocitric acid.

The above mentioned specific preferred starting compounds are aliphatic tricarboxylic acids having a structure according to formula (I), esters thereof, anhydrides thereof, and salts thereof

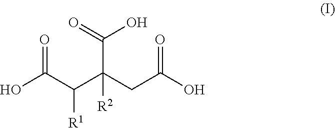

wherein
(i) R¹═H; R²═H (tricarballylic acid), or
(ii) R¹═OH; R²═H (isocitric acid) or
(iii) R¹═H; R²═OH (citric acid) or
(iv) R¹, R² together designate a double bond (aconitic acid).

Thus, the starting compound or at least one of said starting compounds may be selected from the group consisting of
citric acid,
isocitric acid,
aconitic acid,
tricarballylic acid,
esters of acids selected from the group consisting of citric acid, isocitric acid, aconitic acid and tricarballylic acid,
anhydrides of acids selected from the group consisting of citric acid, isocitric acid, aconitic acid and tricarballylic acid,
and salts of acids selected from the group consisting of citric acid, isocitric acid, aconitic acid and tricarballylic acid.

Among these starting compounds, citric acid, isocitric acid, aconitic acid and tricarballylic acid are preferred.

For instance, the starting material comprises or consists of one, two or more starting compounds selected from the group consisting of citric acid, isocitric acid, aconitic acid, tricarballylic acid, esters thereof, anhydrides thereof, and salts thereof. Preferably, all of said starting compounds are selected from the group consisting of citric acid, isocitric acid, aconitic acid, tricarballylic acid, esters thereof, anhydrides thereof, and salts thereof.

A specially preferred starting compound is citric acid. For instance, the starting material comprises or consists of citric acid. Preferably, the starting material consists of citric acid.

The total concentration of starting compounds selected from the group consisting of aliphatic tricarboxylic acids, esters thereof, anhydrides thereof, and salts thereof may be 5 wt.-% or more, preferably 20 wt.-% or more, based on the total amount of the reaction mixture at the beginning of step (iii). The total concentration of all starting compounds selected from the group consisting of aliphatic tricarboxylic acids, esters thereof, anhydrides thereof, and salts thereof may be 60 wt.-% or less, preferably 55 wt.-% or less, based on the total amount of the reaction mixture at the beginning of step (iii).

Herein, the total amount of the reaction mixture at the beginning of step (iii) is the sum of the amount of the starting compounds and the solvent having a having a dielectric constant above the dielectric constant of n-butanol.

Preferably, the total concentration of starting compounds selected from the group consisting of aliphatic tricarboxylic acids, esters thereof, anhydrides thereof and salts thereof is 5 wt.-% or more and 60 wt.-% or less, based on the total amount of the reaction mixture at the beginning of step (iii). More preferably, the total concentration of starting compounds selected from the group consisting of aliphatic tricarboxylic acids, esters thereof, anhydrides thereof, and salts thereof is 20 wt.-% or more and below 55 wt.-% or less, based on the total amount of the reaction mixture at the beginning of step (iii).

The starting material comprising one, two or more starting compounds selected from the group consisting of aliphatic tricarboxylic acids, esters thereof, anhydrides thereof, and salts thereof, preferably selected from the group consisting of citric acid and aconitic acid, is preferably prepared or isolated from plant material. Thus, the above-defined method has the advantage that starting material obtained from renewable sources may be used.

For instance, the starting material is prepared from plant material by sugar fermentation.

Another potential renewable source for starting materials, especially citric acid and starting compounds obtainable from chemical conversion of citric acid, are citrus fruits.

The product resulting in step (iii) of the above-defined method comprises one or more product compounds selected from the group consisting of aliphatic polyols having three or more hydroxy groups and a total number of carbon atoms of 5 or more. For instance, the product resulting in step (iii) may comprise or consist of one, two or more product compounds selected from the group consisting of aliphatic polyols having three or more hydroxy groups and a total number of carbon atoms in the range of from 5 to 6.

Aliphatic polyols having three hydroxy groups and a total number of carbon atoms of 6 are referred to as C₆-triols. Aliphatic polyols having four hydroxy groups and a total number of carbon atoms of 6 are referred to as C₆-tetrols. Aliphatic polyols having three hydroxy groups and a total number of carbon atoms of 5 are referred to as C₅-triols.

Preferably, the product compounds are compounds selected from the group consisting of
aliphatic polyols having three or four hydroxy groups and a total number of carbon atoms of 6
aliphatic polyols having three hydroxy groups and a total number of carbon atoms of 5.

For instance, the product resulting in step (iii) comprises or consists of one, two or more product compounds selected from the group consisting of aliphatic polyols having three or four hydroxy groups and a total number of carbon atoms of 6 and aliphatic polyols having three hydroxy groups and a total number of carbon atoms of 5. Preferably, all product compounds are compounds selected from the group consisting of aliphatic polyols having three or four hydroxy groups and a total number of carbon atoms of 6 and aliphatic polyols having three hydroxy groups and a total number of carbon atoms of 5.

Preferred product compounds selected from the group consisting of aliphatic polyols having three or four hydroxy groups and a total number of carbon atoms of 6 are 3-(hydroxymethyl)pentane-1,3,5-triol, propane-1,2,3-trimethanol and 3-(hydroxymethyl)-2-pentene-1,5-diol. A preferred product compound selected from the group consisting of aliphatic polyols having three hydroxy groups and a total number of carbon atoms of 5 is 1,3,5-pentanetriol.

In certain preferred cases the product resulting in step (iii) comprises two or more product compounds selected from the group consisting of 3-(hydroxymethyl)pentane-1,3,5-triol, propane-1,2,3-trimethanol, 3-(hydroxymethyl)-2-pentene-1,5-diol, and 1,3,5-pentanetriol.

From starting compounds selected from the group consisting of citric acid (exemplarily shown below), esters thereof, salts thereof and anhydrides thereof, the product compounds 3-(hydroxymethyl)pentane-1,3,5-triol, propane-1,2,3-trimethanol, 3-(hydroxymethyl)-2-pentene-1,5-diol, and 1,3,5-pentanetriol may be obtained by the method according to the present invention:

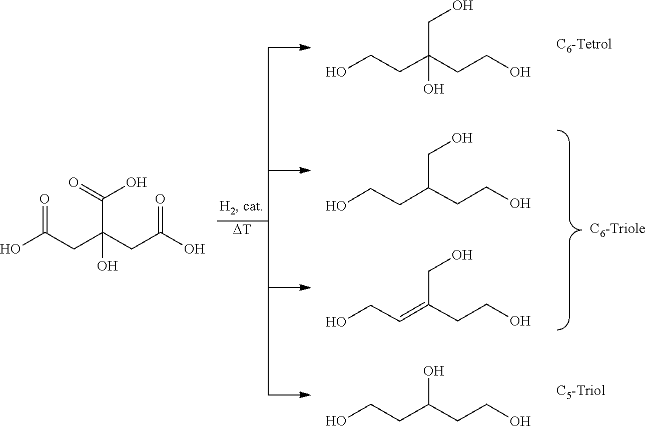

From starting compounds selected from the group consisting of aconitic acid (exemplarily shown below), esters thereof, salts thereof and anhydrides thereof, the product compounds propane-1,2,3-trimethanol and 3-(hydroxymethyl)-2-pentene-1,5-diol may be obtained by the method according to the present invention:

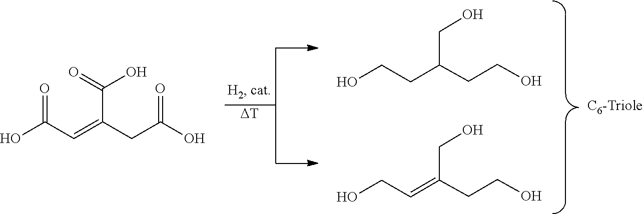 [SMILES: O=C(O)/C=C(/CC(=O)O)C(=O)O ; "DT" ; OC/C=C(\CO)CCO ; OCCC(CO)CCO]

From starting compounds selected from the group consisting of tricarballylic acid, esters thereof, salts thereof and anhydrides thereof, the product compound propane-1,2,3-trimethanol may be obtained by the method according to the present invention.

From starting compounds selected from the group consisting of isocitric acid (exemplarily shown below), esters thereof, salts thereof and anhydrides thereof, the following product compounds may be obtained by the method according to the present invention:

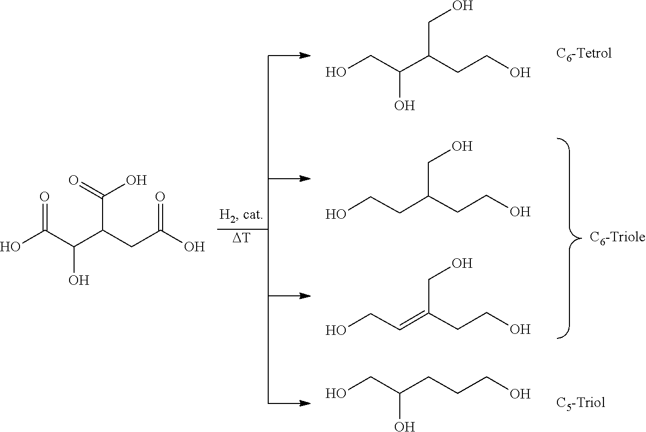

In the product resulting in step (iii) the total amount of aliphatic polyols having three or more hydroxy groups and a total number of carbon atoms in the range of from 5 to 6 may be 30 wt.-% or more, preferably 50 wt.-% or more, or in the range of from 40 to 95 wt.-%, in each case based on the total amount of residual starting compounds and product compounds obtained by chemical conversion of the one or more starting compounds, including product compounds which are not aliphatic polyols having three or more hydroxy groups and a total number of carbon atoms in the range of from 5 to 6. It is understood that a certain amount of product compounds which are not aliphatic polyols having three or more hydroxy groups and a total number of carbon atoms in the range of from 5 to 6 may result from unavoidable side reactions.

Preferably, in the product resulting in step (iii) the total amount of product compounds selected from the group consisting of 3-(hydroxymethyl)pentane-1,3,5-triol, propane-1,2,3-trimethanol, 3-(hydroxymethyl)-2-pentene-1,5-diol, and 1,3,5-pentanetriol, is above 30 wt. %, preferably above 50 wt.-%, or in the range of from 40 to 95 wt.-%, in each case based on the total amount of residual starting compounds and product compounds obtained by chemical conversion of the one or more starting compounds, including product compounds which are not aliphatic polyols having three or more hydroxy groups and a total number of carbon atoms in the range of from 5 to 6. It is understood that a certain amount of product compounds which are not aliphatic polyols having three or more hydroxy groups and a total number of carbon atoms in the range of from 5 to 6 may result from unavoidable side reactions.

When an aliphatic tricarboxylic acid or a salt thereof or an anhydride thereof is used as a starting compound, it is preferably chemically converted in step (iii) so that predominantly the number of carbon atoms present in said starting compound is maintained so that the resulting aliphatic polyol(s) has/have the same number of carbon atoms as said starting compound, or the number of carbon atoms present in said starting compound is reduced by one so that the resulting aliphatic polyol(s) has/have one carbon atom less than said starting compound. Thus, predominantly not more than one carbon atom of the starting compound is lost by decarboxylation. In certain cases, said starting compound is chemically converted in step (iii) so that predominantly the number of carbon atoms present in said starting compound is maintained so that the resulting aliphatic polyol(s) has/have the same number of carbon atoms as said starting compound. In this case, predominantly no carbon atom of the starting compound is lost by decarboxylation. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an aliphatic tricarboxylic acid or a salt thereof or an anhydride thereof is used as a starting compound, it is preferably chemically converted in step (iii) so that predominantly the number of carbon-carbon single bonds present in said starting compound is maintained so that the resulting aliphatic polyol(s) has/have the same number of carbon-carbon single bonds as said starting compound, or one carbon-carbon single bond present in said starting compound is converted into a carbon-carbon double bond so that the resulting aliphatic polyol(s) has/have one carbon-carbon single bond less than said starting compound. Thus, predominantly not more than one carbon-carbon single bond of the starting compound is converted into a carbon-carbon double bond by dehydratation. In certain cases, said starting compound is chemically converted in step (iii) so that predominantly the number of carbon-carbon single bonds present in said starting compound is maintained so that the resulting aliphatic polyol(s) has/have the same number of carbon-carbon single bonds as said starting compound. In this case, predominantly no carbon-carbon single bond of the starting compound is converted into a carbon-carbon double bond by dehydratation. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an alpha-beta-unsaturated aliphatic tricarboxylic acid or a salt thereof or an anhydride thereof is used as a starting compound, it is preferably chemically converted in step (iii) so that predominantly the number of carbon-carbon single bonds present in said starting compound is maintained so that the resulting aliphatic polyol(s) has/have the same number of carbon-carbon single bonds as said starting compound, or the carbon-carbon double bond present in said starting compound is converted into a carbon-carbon single bond by hydrogenation so that the resulting aliphatic polyol(s) has/have one carbon-carbon single bond more than said starting compound. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an ester of an aliphatic tricarboxylic acid is used as a starting compound, it is preferably chemically converted in step (iii) so that predominantly the number of carbon atoms present in the aliphatic tricarboxylic acid corresponding to said ester is maintained so that the resulting aliphatic polyol(s) has/have the same number of carbon atoms as said aliphatic tricarboxylic acid corresponding to said ester, or the number of carbon atoms present in the aliphatic tricarboxylic acid corresponding to said ester is reduced by one so that the resulting aliphatic polyol(s) has/have one carbon atom less than the aliphatic tricarboxylic acid corresponding to said ester. Thus, predominantly not more than one carbon atom of the aliphatic tricarboxylic acid corresponding to the ester used as the starting compound is lost by decarboxylation. In certain cases, said ester is chemically converted in step (iii) so that predominantly the number of carbon atoms present in the aliphatic tricarboxylic acid corresponding to said ester is maintained so that the resulting aliphatic polyol(s) has/have the same number of carbon atoms as said aliphatic tricarboxylic acid corresponding to said ester. In this case, predominantly no carbon atom of the aliphatic tricarboxylic acid corresponding to the ester used as the starting compound is lost by decarboxylation. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an ester of an aliphatic tricarboxylic acid is used as a starting compound, it is preferably chemically converted in step (iii) so that predominantly the number of carbon-carbon single bonds present in the aliphatic tricarboxylic acid corresponding to said ester is maintained so that the resulting aliphatic polyol(s) has/have the same number of carbon-carbon single bonds as said aliphatic tricarboxylic acid corresponding to said ester, or one carbon-carbon single bond present in the aliphatic tricarboxylic acid corresponding to said ester is converted into a carbon-carbon double bond so that the resulting aliphatic polyol(s) has/have one carbon-carbon single bond less than said aliphatic tricarboxylic acid corresponding to said ester. Thus, predominantly not more than one carbon-carbon single bond of the aliphatic tricarboxylic acid corresponding to said ester is converted into a carbon-carbon double bond by dehydratation. In certain cases, said ester is chemically converted in step (iii) so that predominantly the number of carbon-carbon single bonds present in the aliphatic tricarboxylic acid corresponding to said ester is maintained so that the resulting aliphatic polyol(s) has/have the same number of carbon-carbon single bonds as said aliphatic tricarboxylic acid corresponding to said ester. In this case, predominantly no carbon-carbon single bond of the aliphatic tricarboxylic acid corresponding to said ester is converted into a carbon-carbon double bond by dehydratation. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an ester of an alpha-beta unsaturated aliphatic tricarboxylic acid is used as a starting compound, it is preferably chemically converted in step (iii) so that predominantly the number of carbon-carbon single bonds present in the alpha-beta unsaturated aliphatic tricarboxylic acid corresponding to said ester is maintained so that the resulting aliphatic polyol(s) has/have the same number of carbon-carbon single bonds as said alpha-beta unsaturated aliphatic tricarboxylic acid corresponding to said ester, or the carbon-carbon double bond present in the alpha-beta unsaturated aliphatic tricarboxylic acid corresponding to said ester is converted into a carbon-carbon single bond so that the resulting aliphatic polyol(s) has/have one carbon-carbon single bond more than said alpha-beta unsaturated aliphatic tricarboxylic acid corresponding to said ester. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When citric acid or a salt thereof or the anhydride thereof is used as a starting compound, it is preferably chemically converted in step (iii) so that predominantly the number of carbon atoms present in citric acid is maintained so that the resulting aliphatic polyol(s) has/have the same number of carbon atoms as citric acid, or the number of carbon atoms present in citric acid is reduced by one so that the resulting aliphatic polyol(s) has/have one carbon atom less than citric acid. Thus, predominantly not more than one carbon atom of citric acid is lost by decarboxylation. In certain cases, citric acid is chemically converted in step (iii) so that predominantly the number of carbon atoms present in citric acid is maintained so that the resulting aliphatic polyol(s) has/have the same number of carbon atoms as citric acid. In this case, predominantly no carbon atom of citric acid is lost by decarboxylation. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When citric acid or a salt thereof or the anhydride thereof is used as a starting compound, it is preferably chemically converted in step (iii) so that predominantly the number of carbon-carbon single bonds present in citric acid is maintained so that the resulting aliphatic polyol(s) has/have the same number of carbon-carbon single bonds as citric acid, or one carbon-carbon single bond present in citric acid is converted into a carbon-carbon double bond by dehydratation so that the resulting aliphatic polyol(s) has/have one carbon-carbon single bond less than citric acid. In certain cases, said starting compound is chemically converted in step (iii) so that predominantly the number of carbon-carbon single bonds present in citric acid is maintained so that the resulting aliphatic polyol(s) has/have the same number of carbon-carbon single bonds as citric acid. In this case, predominantly no dehydratation of citric acid occurs. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an ester of citric acid is used as a starting compound, it is preferably chemically converted in step (iii) so that predominantly the number of carbon atoms present in citric acid is maintained so that the resulting aliphatic polyol(s) has/have the same number of carbon atoms as citric acid, or the number of carbon atoms present in citric acid is reduced by one so that the resulting aliphatic polyol(s) has/have one carbon atom less than citric acid. Thus, predominantly not more than one carbon atom of citric acid is lost by decarboxylation. In certain cases, said starting compound is chemically converted in step (iii) so that predominantly the number of carbon atoms present in citric acid is maintained so that the resulting aliphatic polyol(s) has/have the same number of carbon atoms as citric acid. In this case, predominantly no carbon atom of citric acid is lost by decarboxylation. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

When an ester of citric acid is used as a starting compound, it is preferably chemically converted in step (iii) so that predominantly the number of carbon-carbon single bonds present in citric acid is maintained so that the resulting aliphatic polyol(s) has/have the same number of carbon-carbon single bonds as citric acid, or one carbon-carbon single bond present in citric acid is converted into a carbon-carbon double bond by dehydratation so that the resulting aliphatic polyol(s) has/have one carbon-carbon single bond less than citric acid. In certain cases, said starting compound is chemically converted in step (iii) so that predominantly the number of carbon-carbon single bonds present in citric acid is maintained so that the resulting aliphatic polyol(s) has/have the same number of carbon-carbon single bonds as citric acid. In this case, predominantly no dehydratation of citric acid occurs. Herein, predominantly means that more than 50% by weight of the above-defined product (a) fulfills the above-defined condition. Preferably, more than 60%, more preferably more than 70% by weight of the product (a) fulfills the above-defined condition. Herein, the total weight of the product (a) is the sum of the weights of the aliphatic polyols having three or more hydroxy groups and a total number of carbon atoms of 5 or more, residual starting compounds, and product compounds formed by side reactions which are not aliphatic polyols having three or more hydroxy groups and a total number of carbon atoms in the range of from 5 to 6.

In step (ii) of the above-defined method, a solvent having a dielectric constant above the dielectric constant of n-butanol is prepared or provided. Thus, the solvent has a higher polarity than n-butanol. Preferred solvents are protic. Such solvents have a sufficient solubility for the above-defined starting compounds.

The solvent provided or prepared in step (ii) may comprise one or more constituents selected from the group consisting of water, methanol, ethanol, n-propanol, iso-propanol, ethylene glycols, propylene glycols, and cyclic ethers in such proportions that a dielectric constant above the dielectric constant of n-butanol results. Typical cyclic ethers are tetrahydrofurane (THF), tetrahydropyrane (THP), and dioxanes.

Preferably the solvent is selected from the group consisting of
water,
   and
aqueous mixtures comprising water in an amount of more than 50 wt.-%, preferably more than 70 wt.-%, more preferably more than 90 wt.-%, based on the total amount of the solvent.

In said aqueous mixtures, one or more of methanol, ethanol, n-propanol, iso-propanol, ethylene glycols, propylene glycols, cyclic ethers may be admixed to water.

In step (iii) of the above-defined method, said one or more starting compounds provided or prepared in step (i) are chemically converted into one or more of the above-defined product compounds at a temperature in the range of from 80° C. to 155° C., preferably at temperatures in the range of from 120° C. to 155° C.

Depending on the reaction temperature, chemical conversion of starting compounds selected from the group consisting of aliphatic tricarboxylic acids, esters thereof, anhydrides thereof, and salts thereof at a partial pressure of hydrogen in the range of from 15 MPa to 30 MPa in the presence of a given heterogeneous hydrogenation catalyst may result either in lactones (not according to the invention, cf. FIG. 1) or in aliphatic polyols having three or more hydroxy groups and a total number of carbon atoms of 5 or more or mixtures of both kinds of products. Therefore, in step (iii), the reaction temperature is preferably chosen so as to selectively prepare said aliphatic polyols having three or more hydroxy groups and a total number of carbon atoms of 5 or more instead of lactones.

Step (iii) of the above-defined method may be conducted for a period of 30 hours or more, preferably 60 hours or more. Step (iii) of the above-defined method may be conducted for a period of 200 hours or less, preferably 120 hours or less, more preferably 96 hours or less. Preferably, step (iii) of the above-defined method is conducted for a period of 30 hours or more, and 200 hours or less. More preferably, step (iii) of the above-defined method is conducted for a period of 60 hours or more, and 120 hours or less, preferably 96 hours or less.

In step (iii) of the above-defined method, said one or more starting compounds provided or prepared in step (i) are chemically converted into one or more of the above-defined product compounds in the presence of a heterogeneous hydrogenation catalyst comprising one or more metals selected from the group consisting of Mn, Re, Fe, Ru, and Os.

Preferred heterogeneous hydrogenation catalysts comprise or consist of one or more metals selected from the group consisting of Mn, Re, Fe, Ru, and Os in a total amount of 90 wt.-% or more, preferably 95 wt.-% or more, based on the total amount of the heterogeneous hydrogenation catalyst. Herein preferably the amount of Ru is 90 wt.-% or more, preferably 95 wt.-% or more, based on the total amount of the heterogeneous hydrogenation catalyst.

Other preferred heterogeneous hydrogenation catalysts comprise or consist of one or more metals selected from the group consisting of Mn, Re, Fe, Ru, and Os in a total amount of 10 wt.-% or more, based on the total amount of the heterogeneous hydrogenation catalyst, in combination with one or more metals selected from the group consisting of Co, Rh, Ir, Ni, Pd, Pt, Cu, Ag and Au, wherein the total amount of metals selected from the group consisting of Mn, Re, Fe, Ru, Os, Co, Rh, Ir, Ni, Pd, Pt, Cu, Ag and Au is 98 wt.-% or more, based on the total amount of the heterogeneous hydrogenation catalyst. Preferred are catalysts comprising or consisting of one or both of Re and Ru in combination with one or both of Pd and Pt, wherein the total amount of metals selected from the group consisting of Re, Rh, Pd and Pt is 98 wt.-% or more, based on the total amount of the heterogeneous hydrogenation catalyst.

It is understood that in each case the catalyst may comprise traces of other metals and of oxides, which are included in the above-mentioned amount of the heterogeneous hydrogenation catalyst.

Preferably, the amount of heterogeneous hydrogenation catalyst relative to the total amount of the starting compounds is in the range of from 0.01 wt % to 5 wt. %, preferably 0.05 wt % to 2.5 wt %.

The heterogeneous hydrogenation catalyst may be supported by a support material (also referred to as a carrier). The weight of the support material is not included in the above-mentioned amount of the heterogeneous hydrogenation catalyst.

The support material of the heterogeneous hydrogenation catalyst is preferably chosen so as to sustain the hydrothermal stress resulting from the simultaneous presence of heat, water, acid and hydrogenolytic conditions during step (iii) of the above-defined method.

Preferably, the support material is selected from the group consisting of metal oxides, zeolites and carbon-based materials, preferably Al₂O₃, ZrO₂, TiO₂, SiC, carbon black and PTFE. Combinations of different support materials are possible, e.g. PTFE-supported carbon black.

In supported catalysts, preferably the total amount of heterogeneous hydrogenation catalyst is in the range of from 0.1 wt.-% to 10 wt.-%, preferably 0.5 wt.-% to 5 wt.-%, relative to the sum of the weight of the heterogeneous hydrogenation catalyst and the support material.

Preferred combinations of catalyst metals and support materials are Ru in combination with carbon black, PTFE-supported carbon black, Al₂O₃, or SiC, and Re/Pt in combination with carbon black.

After step (iii), the supported heterogeneous hydrogenation catalyst may be recovered, preferably be means of filtration.

The above-defined method may comprise one or more additional steps which are conducted after above-defined step (iii).

In an additional step conducted after above-defined step (iii), the solvent used in step (iii) may be removed by evaporation. Evaporation of the solvent may be carried out by any suitable method. For instance, evaporation of the solvent may be carried out by freeze-drying.

In an additional step conducted after above-defined step (iii), one or more of said product compounds selected from the group consisting of aliphatic polyols having three or more hydroxy groups and a total number of carbon atoms of 5 or more present in the product resulting from step (iii) may be chemically converted to give a product (b) comprising one or more reaction products of one or more of said compounds selected from the group consisting of aliphatic polyols having three or more hydroxy groups and a total number of carbon atoms of 5 or more. Herein, the product resulting in step (iii), which comprises one or more product compounds selected from the group consisting of aliphatic polyols having three or more hydroxy groups and a total number of carbon atoms of 5 or more, is an intermediate product, and said intermediate product is chemically converted into a final product comprising one or more reaction products of one or more of said compounds selected from the group consisting of aliphatic polyols having three or more hydroxy groups and a total number of carbon atoms of 5 or more.

In an additional step conducted after above-defined step (iii), one or more additional chemical substances may be added to one or more of said product compounds selected from the group consisting of aliphatic polyols having three or more hydroxy groups and a total number of carbon atoms of 5 or more present in the product resulting from step (iii), so that a reaction mixture results comprising said one or more product compounds selected from the group consisting of aliphatic polyols having three or more hydroxy groups and a total number of carbon atoms of 5 or more. Thus, a reaction mixture is formed which contains
one or more aliphatic polyols having three or more hydroxy groups and a total number of carbon atoms of 5 or more resulting from above-defined step (iii)
one or more additional chemical substances which are not aliphatic polyols having three or more hydroxy groups and a total number of carbon atoms of 5 or more.

Such reaction mixtures are configured and intended for being used for preparing reaction products (for details see below) different from the above-mentioned product comprising one or more product compounds selected from the group consisting of aliphatic polyols having three or more hydroxy groups and a total number of carbon atoms of 5 or more, which results from above-defined step (iii).

In said reaction mixture, said one, two or more product compounds selected from the group consisting of aliphatic polyols having three or more hydroxy groups and a total number of carbon atoms of 5 or more may function as a reactant or as a solvent.

The above-defined method may comprise one, two or all of the above-defined additional steps which are conducted after above-defined step (iii).

As explained above, in case (a) the product of the above-defined method comprises one or more product compounds selected from the group consisting of aliphatic polyols having three or more hydroxy groups and a total number of carbon atoms of 5 or more.

Product (a) may be selected from the group consisting of formulations comprising one or more materials selected from the group consisting of:
peptides,
proteins, wherein preferably the proteins are selected from the group consisting of human serum albumin and bovine serum albumin,
enzymes, wherein preferably the enzymes are selected from the group consisting of lysozymes, proteases, amylases, lipases, mannanases, and cellulases,
microorganisms, wherein preferably the microorganisms are selected from the group consisting of gram-positive bacteria, gram-negative bacteria, spore forming bacteria, fungal spore, mycelia, yeasts,
DNA, RNA and viruses, wherein preferably the viruses are selected from the group consisting of bacteriophages.

Formulations comprising one or more materials selected from the group consisting of peptides, proteins, enzymes, DNA, RNA, viruses and microorganisms are used for a wide variety of different applications including biocatalysis, food sector, feed applications, home care, personal care and agriculture.

In such formulations the product compounds selected from the group consisting of aliphatic polyols having three or more hydroxy groups and a total number of carbon atoms of 5 or more may substitute conventional solvents like 1,2-propanediol, glycerol or sorbitol, and/or may protect and stabilize the materials selected from the group consisting of peptides, proteins, enzymes, DNA, RNA, viruses and microorganisms. More specifically the product compounds selected from the group consisting of aliphatic polyols having three or more hydroxy groups and a total number of carbon atoms of 5 or more may be used as a biostatic agent to avoid proliferation of microorganisms.

Product (a) may be a paint formulation or an adhesive formulation, preferably a paint formulation or an adhesive formulation comprising phenoxyethanol. In such formulations, product compounds selected from the group consisting of aliphatic polyols having three or more hydroxy groups and a total number of carbon atoms of 5 or more may intensify the biocidal effect of phenoxyethanol.

Product (a) may be a reaction mixture, wherein one or more product compounds selected from the group consisting of aliphatic polyols having three or more hydroxy groups and a total number of carbon atoms of 5 or more substitute conventional polyols like 1,1,1-trimethylolpropane (TMP) or pentaerythritol (PETP).

For instance, product (a) is a reaction mixture for converting one or more of said aliphatic polyols having three or more hydroxy groups and a total number of carbon atoms of 5 or more into polyalkoxylates by alkoxylation with ethylene oxide and/or propylene oxide wherein the reaction mixture is prepared after step (iii) of chemically converting said one or more starting compounds.

For instance, product (a) is a reaction mixture for preparing polymers, preferably selected from the group consisting of polyurethanes, polyesters and polyacrylates, wherein the reaction mixture is prepared after step (iii) of chemically converting said one or more starting compounds. In a reaction mixture for preparing polyacrylates, the product compounds selected from the group consisting of aliphatic polyols having three or more hydroxy groups and a total number of carbon atoms of 5 or more may provide encapsulation after suspension polymerization.

For instance, product (a) is a reaction mixture for preparing esters, acyclic ethers or cyclic ethers of said one or more product compounds selected from the group consisting of aliphatic polyols having three or more hydroxy groups and a total number of carbon atoms of 5 or more, wherein the reaction mixture is prepared after step (iii) of chemically converting said one or more starting compounds. Preferred esters are acetyl esters. Acyclic ethers are obtainable by tert-butylization. Cyclic ethers are obtainable by dehydrative cyclization. After further functionalization, said esters, acyclic ethers and cyclic ethers may be potential building blocks for scent and aroma chemistry.

The above-mentioned reaction mixtures may be obtained by adding one or more additional chemical substances to one, two or more of said product compounds selected from the group consisting of aliphatic polyols having three or more hydroxy groups and a total number of carbon atoms of 5 or more in an additional step conducted after step (iii) of chemically converting said one or more starting compounds.

As explained above, in case (b) the product of the above-defined method comprises one or more reaction products of one or more of said compounds selected from the group consisting of aliphatic polyols having three or more hydroxy groups and a total number of carbon atoms of 5 or more.

Product (b) may be selected from the group consisting of products comprising one or more polyalkoxylates, wherein at least one of said polyalkoxylates is prepared from one or more of said product compounds selected from the group consisting of aliphatic polyols having three or more hydroxy groups and a total number of carbon atoms of 5 or more, in an additional step conducted after step (iii) of chemically converting said one or more starting compounds. Such polyalkoxylates may be used as non-ionic foam suppressants and demulsifiers in a wide variety of applications, e.g. home and personal care applications. Polyalkoxylates obtained by the method according to the present invention are expected to have enhanced biodegradability, compared to conventional polyalkoxylates.

Product (b) may be selected from the group consisting of products comprising one or more polymers, preferably selected from the group consisting of polyurethanes and polyesters, wherein at least one of said polymers is prepared from one or more of said product compounds selected from the group consisting of aliphatic polyols having three or more hydroxy groups and a total number of carbon atoms of 5 or more in an additional step conducted after step (iii) of chemically converting said one or more starting compounds.

In a specifically preferred method according to the present invention, a product comprising
(a) one, two, three or all product compounds selected from the group consisting of 3-(hydroxymethyl)pentane-1,3,5-triol, propane-1,2,3-trimethanol, 3-(hydroxymethyl)-2-pentene-1,5-diol and 1,3,5-pentanetriol
   and/or
(b) one or more reaction products of one, two, three or all of said product compounds is prepared by a method comprising at least the following steps:
(i) providing or preparing a starting material comprising one, two or more starting compounds selected from the group consisting of citric acid, esters thereof, anhydrides thereof, and salts thereof,
(ii) providing or preparing a solvent having a dielectric constant above the dielectric constant of n-butanol, wherein said solvent is water or a mixture of water with one selected from the group consisting of methanol and ethylene glycols,
   and
(iii) chemically converting said one, two or more starting compounds provided or prepared in step (i)
   at a temperature in the range of from 120° C. to 155° C.,
   at a partial pressure of hydrogen in the range of from 15 MPa to 30 MPa,
   in said solvent provided or prepared in step (ii), and
   in the presence of a heterogeneous hydrogenation catalyst comprising Ru,
   so that carboxyl groups, carboalkoxy groups, anhydride groups and/or carboxylate groups as present in the one, two or more starting compounds are selectively hydrogenated to the corresponding hydroxy groups so that a product results comprising one, two, three or all product compounds selected from the group consisting of 3-(hydroxymethyl)pentane-1,3,5-triol, propane-1,2,3-trimethanol, 3-(hydroxymethyl)-2-pentene-1,5-diol, and 1,3,5-pentanetriol.

In another specifically preferred method according to the present invention, a product comprising
(a) one or both product compounds selected from the group consisting of propane-1,2,3-trimethanol and 3-(hydroxymethyl)-2-pentene-1,5-diol,
   and/or
(b) one or more reaction products of one or both of said product compounds
   is prepared by a method comprising at least the following steps:
(i) providing or preparing a starting material comprising one, two or more starting compounds selected from the group consisting of aconitic acid, esters thereof, anhydrides thereof, and salts thereof,
(ii) providing or preparing a solvent having a dielectric constant above the dielectric constant of n-butanol, wherein said solvent is water or a mixture of water with one selected from the group consisting of methanol and ethylene glycols,
   and
(iii) chemically converting said one, two or more starting compounds provided or prepared in step (i)
   at a temperature in the range of from 120° C. to 155° C.,
   at a partial pressure of hydrogen in the range of from 15 MPa to 30 MPa,
   in said solvent provided or prepared in step (ii), and
   in the presence of a heterogeneous hydrogenation catalyst comprising Ru,
   so that carboxyl groups, carboalkoxy groups, anhydride groups and/or carboxylate groups as present in the one, two or more starting compounds are selectively hydrogenated to the corresponding hydroxy groups so that a product comprising one or both product compounds selected from the group consisting of propane-1,2,3-trimethanol and 3-(hydroxymethyl)-2-pentene-1,5-diol results.

In a further specifically preferred method according to the present invention, a product comprising
(a) the product compound propane-1,2,3-trimethanol
   and/or
(b) one or more reaction products of said product compound
   is prepared by a method comprising at least the following steps:
(i) providing or preparing a starting material comprising one, two or more starting compounds selected from the group consisting of tricarballylic acid, esters thereof, anhydrides thereof, and salts thereof,
(ii) providing or preparing a solvent having a dielectric constant above the dielectric constant of n-butanol, wherein said solvent is water or a mixture of water with one selected from the group consisting of methanol and ethylene glycols,
   and
(iii) chemically converting said one, two or more starting compounds provided or prepared in step (i)
   at a temperature in the range of from 120° ° C. to 155° C.,
   at a partial pressure of hydrogen in the range of from 15 MPa to 30 MPa,
   in said solvent provided or prepared in step (ii), and
   in the presence of a heterogeneous hydrogenation catalyst comprising Ru,
   so that carboxyl groups, carboalkoxy groups, anhydride groups and/or carboxylate groups as present in the one, two or more starting compounds are selectively hydrogenated to the corresponding hydroxy groups so that a product comprising the product compound propane-1,2,3-trimethanol results.

The present application also relates to a product selected from the group consisting of
formulations comprising one or more materials selected from the group consisting of:
   peptides,
   proteins, wherein preferably the proteins are selected from the group consisting of human serum albumin and bovine serum albumin,
   enzymes, wherein preferably the enzymes are selected from the group consisting of lysozymes, proteases, amylases, lipases, mannanases, and cellulases,
   microorganisms, wherein preferably the microorganisms are selected from the group consisting of gram-positive bacteria, gram-negative bacteria, spore forming bacteria, fungal spore, mycelia, yeasts,
   DNA, RNA and viruses, wherein preferably the viruses are selected from the group consisting of bacteriophages
paint formulations and adhesive formulations, preferably comprising phenoxyethanol,
reaction mixtures for converting one, two or more of said aliphatic polyols having three or more hydroxy groups and a total number of carbon atoms of 5 or more into polyalkoxylates by alkoxylation with ethylene oxide and/or propylene oxide, wherein the reaction mixture is prepared after step (iii),
reaction mixtures for preparing polymers, preferably selected from the group consisting of polyurethanes, polyesters and polyacrylates, wherein the reaction mixture is prepared after step (iii), and
reaction mixtures for preparing esters, acyclic ethers or cyclic ethers of said one, two or more product compounds selected from the group consisting of aliphatic polyols having three or more hydroxy groups and a total number of carbon atoms of 5 or more, wherein the reaction mixture is prepared after step (iii)
   wherein the product comprises one, two or more product compounds selected from the group consisting of aliphatic polyols having three or more hydroxy groups and a total number of carbon atoms in the range of from 5 to 6.

Preferably, an above-defined product comprises aliphatic polyols selected from the group consisting of
aliphatic polyols having three or four hydroxy groups and a total number of carbon atoms of 6, and
aliphatic polyols having three hydroxy groups and a total number of carbon atoms of 5.

Said aliphatic polyols having three or four hydroxy groups and a total number of carbon atoms of 6 are preferably selected from the group consisting of 3-(hydroxymethyl)pentane-1,3,5-triol, propane-1,2,3-trimethanol and 3-(hydroxymethyl)-2-pentene-1,5-diol. A preferred aliphatic polyol having three hydroxy groups and a total number of carbon atoms of 5 is 1,3,5-pentanetriol.

More preferably a product as defined above comprises two or more product compounds selected from the group consisting of 3-(hydroxymethyl)pentane-1,3,5-triol, propane-1,2,3-trimethanol, 3-(hydroxymethyl)-2-pentene-1,5-diol and 1,3,5-pentanetriol.

A product as defined above is obtainable by the above-defined method, preferably by one of the above-defined preferred methods.

The present application also relates to the use of one or more compounds selected from the group consisting of aliphatic tricarboxylic acids, esters thereof, anhydrides thereof, and salts thereof, as starting compound(s) for making
one or more product compounds selected from the group consisting of
   aliphatic polyols having three or four hydroxy groups and a total number of 6 carbon atoms,
   aliphatic polyols having three hydroxy groups and a total number of 5 carbon atoms,
      or
mixtures of such product compounds.

Regarding specific and preferred aliphatic tricarboxylic acids, esters thereof, anhydrides thereof, and salts thereof to be used as starting compounds for making the above-defined product compounds, the same applies as disclosed in the context of the above-defined method. Most preferred is the use of citric acid and aconitic acid as starting compounds for making the above-defined product.

Regarding specific and preferred product compounds, the same applies as disclosed in the context of the above-defined method. Aliphatic polyols having three or four hydroxy groups and a total number of carbon atoms of 6 are preferably selected from the group consisting of 3-(hydroxymethyl)pentane-1,3,5-triol, propane-1,2,3-trimethanol, and 3-(hydroxymethyl)-2-pentene-1,5-diol. A preferred aliphatic polyol having three hydroxy groups and a total number of carbon atoms of 5 is 1,3,5-pentanetriol.

Preferred is the use of one or more compounds selected from the group consisting of aliphatic tricarboxylic acids, esters thereof, anhydrides thereof, and salts thereof, as starting compound(s) for making
product compounds selected from the group consisting of
   aliphatic polyols having three or four hydroxy groups and a total number of 6 carbon atoms, and
   aliphatic polyols having three hydroxy groups and a total number of 5 carbon atoms,
      or
mixtures of such product compounds
   in quantities of equal to or more than 100 kg, more preferably equal to or more than 500 kg and even more preferably equal to or more than 1000 kg per batch.

Most preferably, the above-defined preferred compounds selected from the group consisting of aliphatic tricarboxylic acids, esters thereof, anhydrides thereof, and salts thereof are used as starting compound(s) in the method as defined above.

The present application also relates to the use of a heterogeneous hydrogenation catalyst
comprising one or more metals selected from the group consisting of Mn, Re, Fe, Ru, and Os, in a total amount of 90 wt.-% or more, preferably 95 wt.-% or more, based on the total amount of the heterogeneous hydrogenation catalyst, wherein preferably the amount of Ru is 90 wt.-% or more, preferably 95 wt.-% or more, based on the total amount of the heterogeneous hydrogenation catalyst or
comprising one or more metals selected from the group consisting of Mn, Re, Fe, Ru, and Os in a total amount of 10 wt.-% or more, based on the total amount of the heterogeneous hydrogenation catalyst, in combination with one or more metals selected from the group consisting of Co, Rh, Ir, Ni, Pd, Pt, Cu, Ag and Au, preferably Re and/or Ru in combination with Pd and/or Pt, wherein the total amount of metals selected from the group consisting of Mn, Re, Fe, Ru, Os, Co, Rh, Ir, Ni, Pd, Pt, Cu, Ag and Au is 98 wt.-% or more, based on the total amount of the heterogeneous hydrogenation catalyst,
   in a method of preparing a product comprising one, two or more product compounds selected from the group consisting of aliphatic polyols having three or more hydroxy groups and a total number of carbon atoms of 5 or more, wherein the method comprises chemically converting one, two or more starting compounds selected from the group consisting of aliphatic tricarboxylic acids, esters thereof, anhydrides thereof, and salts thereof, so that a product results comprising one, two or more product compounds selected from the group consisting of aliphatic polyols having three or more hydroxy groups and a total number of carbon atoms of 5 or more.

Regarding specific and preferred heterogeneous hydrogenation catalysts, the same applies as disclosed in the context of the above-defined method. Regarding specific and preferred aliphatic tricarboxylic acids, esters thereof, anhydrides thereof, and salts thereof to be converted in the method using the above-defined catalyst, the same applies as disclosed in the context of the above-defined method. Most preferred is the use of citric acid and aconitic acid for making the above-defined product.

Regarding specific and preferred product compounds, the same applies as disclosed in the context of the above-defined method. Aliphatic polyols having three or four hydroxy groups and a total number of carbon atoms of 6 are preferably selected from the group consisting of 3-(hydroxymethyl)pentane-1,3,5-triol, propane-1,2,3-trimethanol, and 3-(hydroxymethyl)-2-pentene-1,5-diol. A preferred aliphatic polyol having three hydroxy groups and a total number of carbon atoms of 5 is 1,3,5-pentanetriol.

Regarding specific and preferred reaction parameters (temperature, hydrogen partial pressure, duration of chemically conversion, solvent), the same applies as disclosed in the context of the above-defined method. Most preferably, the above-defined heterogeneous catalyst is used in the method as defined above.

### EXAMPLES

The following examples according to the present invention are meant to further explain and illustrate the present invention without limiting its scope.

FIG. 1 shows formation of aliphatic polyols having three or more hydroxy groups and a total number of carbon atoms of 5 or more from citric acid as well as products of possible side reactions of citric acid.

Hydrogenation of citric acid dissolved in water was carried out as follows:

The heterogeneous hydrogenation catalyst (type of catalyst, type of support material and amount as given in table 1) was added to a solution of the starting compound citric acid monohydrate in water as the solvent (citric acid concentration and amount of solution given in table 1) in an autoclave (optionally with a catalyst basket). The reaction vessel was closed and then rinsed twice with nitrogen gas (0.5 MPa). Then stirring (700 U/min) and initial hydrogen pressure (5 MPa) were applied. The reaction mixture was heated up to the temperature given in table 1, and the hydrogen pressure was increased up to the value given in table 1. The reaction mixture was stirred under these conditions for the time period given in table 1, then cooled to room temperature and rinsed twice with nitrogen gas (0.5 MPa). Afterwards, the catalyst was filtered off when no catalyst basket had been used. The solvent (water) was removed by evaporation. The resulting oil was analyzed by gas chromatography and HPLC. The experimental parameters and results of the examples with citric acid are compiled in table 1.

Hydrogenation of aconitic acid dissolved in water was carried out as follows:

The heterogeneous hydrogenation catalyst (type of catalyst, type of support material and amount as given in table 2) was added to a solution of the starting compound aconitic acid in water as the solvent (aconitic acid concentration and amount of solution given in table 2) in an autoclave (optionally with a catalyst basket). The reaction vessel was closed and then rinsed twice with nitrogen gas (0.5 MPa). Then stirring (700 U/min) and initial hydrogen pressure (5 MPa) were applied. The reaction mixture was heated up to the temperature given in table 2, and the hydrogen pressure was increased up to the value given in table 2. The reaction mixture was stirred under these conditions for the time period given in table 2, then cooled to room temperature and rinsed twice with nitrogen gas (0.5 MPa). Afterwards, the catalyst was filtered off when no catalyst basket had been used. The solvent (water) was removed by evaporation. The resulting oil was analyzed by gas chromatography and HPLC. The experimental parameters and results of the examples with aconitic acid are compiled in table 2.

Hydrogenation of tricarballylic acid dissolved in water was carried out as follows:

The heterogeneous hydrogenation catalyst (type of catalyst, type of support material and amount as given in table 3) was added to a solution of the starting compound tricarballylic acid in water as the solvent (tricarballylic acid concentration and amount of solution given in table 3) in an autoclave (optionally with a catalyst basket). The reaction vessel was closed and then rinsed twice with nitrogen gas (0.5 MPa). Then stirring (700 U/min) and initial hydrogen pressure (5 MPa) were applied. The reaction mixture was heated up to the temperature given in table 3, and the hydrogen pressure was increased up to the value given in table 3. The reaction mixture was stirred under these conditions for the time period given in table 3, then cooled to room temperature and rinsed twice with nitrogen gas (0.5 MPa). Afterwards, the catalyst was filtered off when no catalyst basket had been used. The solvent (water) was removed by evaporation. The resulting oil was analyzed by gas chromatography and HPLC. The experimental parameters and results of the examples with tricarballylic acid are compiled in table 3.

In the tables,
C₆-tetrol is 3-(hydroxymethyl)pentane-1,3,5-triol
C₆-triols include both propane-1,2,3-trimethanol and 3-(hydroxymethyl)-2-pentene-1,5-diol (except for tricarballylic acid, see above)
C₅-triol is 1,3,5-pentanetriol.

TABLE 1
Parameters and Results of Examples with Citric Acid

| | weight of | conc. of | amount | amount | | | | | | | | | |
|---|---|---|---|---|---|---|---|---|---|---|---|---|---|
| | catalyst relative | citric | of | of | | | | Composition of resulting oil | | | | | |
| | to the weight | acid | aq. citric | catalyst + | | | | | | | | C₆- | |
| | of catalyst + | dissolved | acid | support | | | | Citric | C₆- | C₆- | C₅- | lac- | |
| | support | in H₂O | solution | material | time | p (H₂) | T | Acid | Tetrol | Triols | Triol | tones | Others |
| No. | material | [wt %] | [g] | [g] | [h] | [MPa] | [° C.] | [wt %] | [wt %] | [wt %] | [wt %] | [wt %] | [wt %] |
| 1 | 5 wt.-% Ru/C | 54 | 100 | 10 | 48 | 18 | 140 | — | 19 | 30 | 7 | — | 44 |
| 2 | 5 wt.-% Ru/C | 54 | 100 | 10 | 72 | 18 | 120 | — | 32 | 13 | 23 | — | 32 |
| 3 | 5 wt.-% Re—Pt(1:1)/C | 54 | 100 | 10 | 72 | 27.5 | 120 | 5 | 24 | 31 | — | 7 | 33 |
| 4 | 1.25 wt.-% Ru—Pd | 54 | 100 | 10 | 72 | 27.5 | 120 | 16 | — | 41 | 4 | 22 | 16 |
| | (ratio Ru/Pd 1:4)/ZrO₂ | | | | | | | | | | | | |
| 5 | 5 wt.-% Ru/C | 50 | 100 | 10 | 72 | 27.5 | 120 | — | 37 | 27 | 6 | — | 30 |
| 6 | 5% Ru/C | 54 | 100 | 10 | 72 | 27.5 | 120 | — | 31 | 32 | 2 | — | 35 |
| 7 | 0.5 wt % Ru/C/PTFE | 57 | 50 | 3.5 | 72 | 27.5 | 120 | <1 | 18 | 26 | | — | 56 |
| | (Ratio carbon- | | | | | | | | | | | | |
| | supported | | | | | | | | | | | | |
| | Ru/PTFE = 1:9) | | | | | | | | | | | | |
| 8 | 5 wt % Ru/Al₂O₃ | 54 | 100 | 10 | 72 | 27.5 | 120 | — | 6 | 10 | 29 | 8 | 47 |
| 9 | 3 wt % Ru/SiC | 54 | 100 | 10 | 72 | 27.5 | 150 | — | — | 26 | 6 | — | 68 |
| 10 | 5 wt % Ru/C | 54 | 100 | 10 | 120 | 25.0 | 100 | — | 7 | 22 | 2 | 39 | 30 |
| 11 | 5 wt % Ru/ZrO₂ | 54 | 100 | 10 | 72 | 27.5 | 120 | — | 20 | 29 | 6 | 3 | 42 |

TABLE 2
Parameters and Results of Examples with Aconitic Acid

| | Weight of | conc. of | amount of | | | | | | | | | |
|---|---|---|---|---|---|---|---|---|---|---|---|---|
| | catalyst relative | aconitic | aq. | amount of | | | | | | | | |
| | to the weight | acid | aconitic | catalyst + | | | | Composition of resulting oil | | | | |
| | of catalyst + | dissolved | acid | support | | | | Aconitic | | | C₆-lac- | |
| | support | in H₂O | solution | material | time | p (H₂) | T | Acid | C₆-Triols | C₅-Diol | tones | Others |
| No. | material | [wt %] | [g] | [g] | [h] | [MPa] | [° C.] | [wt %] | [wt %] | [wt %] | [wt %] | [wt %] |
| 1 | 5 wt % Ru/C | 25 | 120 | 10 | 12 | 27.5 | 120 | 1 | 69 | — | — | 30 |
| 2 | 5 wt % Ru/C | 28 | 106 | 6 | 24 | 18 | 140 | 1 | 49 | 30 | — | 20 |

TABLE 3
Parameters and Results of Examples with Tricarballylic Acid

| | | conc. of | amount of | | | | | | | | | |
|---|---|---|---|---|---|---|---|---|---|---|---|---|
| | weight of | tricarb- | aq. | | | | | | | | | |
| | catalyst relative | allylic | tricarb- | amount of | | | | Composition of resulting oil | | | | |
| | to the weight | acid | allylic | catalyst + | | | | Tricarb- | | | | |
| | of catalyst + | dissolved | acid | support | | | | allylic | | | C₆-lac- | |
| | support | in H₂O | solution | material | time | p (H₂) | T | Acid | C₆-Triols | C₅-Diol | tones | Others |
| No. | material | [wt %] | [g] | [g] | [h] | [MPa] | [° C.] | [wt %] | [wt %] | [wt %] | [wt %] | [wt %] |
| 1 | 5 wt % Ru/C | 25 | 120 | 12 | 12 | 18 | 140 | — | 50 | 29 | — | 21 |
| 2 | 5 wt % Ru/C | 25 | 100 | 5 | 72 | 27.5 | 120 | — | 48 | 29 | — | 23 |

1. A method of preparing a product comprising
(a) one, two, or more product compounds selected from the group consisting of aliphatic polyols having three or more hydroxy groups and a total number of carbon atoms in the range of from 5 to 6
or
(b) one or more reaction products of said one, two, or more compounds selected from the group consisting of aliphatic polyols having three or more hydroxy groups and a total number of carbon atoms in the range of from 5 to 6,
comprising:
(i) providing or preparing a starting material comprising a starting compound selected from the group consisting of aliphatic tricarboxylic acids having a total number of 6 carbon atoms, esters thereof, anhydrides thereof, and salts thereof,
(ii) providing or preparing a solvent having a dielectric constant above the dielectric constant of n-butanol,
and
(iii) chemically converting said starting compound provided or prepared in step (i)
at a temperature in the range of from 80° C. to 155° C.,
at a partial pressure of hydrogen in the range of from 15 MPa to 30 MPa,
in said solvent provided or prepared in step (ii), and
in the presence of a heterogeneous hydrogenation catalyst comprising one or more metals selected from the group consisting of Mn, Re, Fe, Ru, and Os,
so that carboxyl groups, carboalkoxy groups, anhydride groups, and/or carboxylate groups as present in the starting compound are selectively hydrogenated to the corresponding hydroxy groups so that a product results comprising one, two, or more product compounds selected from the group consisting of aliphatic polyols having three or more hydroxy groups and a total number of carbon atoms in the range of from 5 to 6.

2. The method according to claim 1, wherein the starting compound
is selected from the group consisting of alpha-functionalized aliphatic tricarboxylic acids, esters thereof, anhydrides thereof, and salts thereof,
or
is selected from the group consisting of alpha-beta-unsaturated aliphatic tricarboxylic acids, esters thereof, anhydrides thereof, and salts thereof,
or
is selected from the group consisting of tricarballylic acid, esters thereof, anhydrides thereof, and salts thereof.

3. The method according to claim 1,
wherein the product resulting in step (iii) comprises one, two, or more product compounds selected from the group consisting of
aliphatic polyols having three or four hydroxy groups and a total number of 6 carbon atoms,
and
aliphatic polyols having three hydroxy groups and a total number of 5 carbon atoms.

4. The method according to claim 1, wherein the solvent having a dielectric constant above the dielectric constant of n-butanol provided or prepared in step (ii)
comprises one or more constituents selected from the group consisting of water, methanol, ethanol, n-propanol, iso-propanol, ethylene glycols, propylene glycols and cyclic ethers.

5. The method according to claim 1, wherein the temperature in step (iii) is
in the range of from 120° C. to 155° C.
and/or
chosen so as to selectively prepare said aliphatic polyols instead of lactones.

6. The method according to claim 1 wherein the heterogeneous hydrogenation catalyst
comprises one or more metals selected from the group consisting of Mn, Re, Fe, Ru, and Os in a total amount of 90 wt.-% or more, based on the weight of the total amount of the heterogeneous hydrogenation catalyst,
or
one or more metals selected from the group consisting of Mn, Re, Fe, Ru, and Os in a total amount of 10 wt.-% or more, based on the total amount of the heterogeneous hydrogenation catalyst, in combination with one or more metals selected from the group consisting of Co, Rh, Ir, Ni, Pd, Pt, Cu, Ag, and Au, wherein the total amount of metals selected from the group consisting of Mn, Re, Fe, Ru, Os, Co, Rh, Ir, Ni, Pd, Pt, Cu, Ag, and Au is 98 wt.-% or more, based on the total amount of the heterogeneous hydrogenation catalyst.

7. The method according to claim 6, wherein the heterogeneous hydrogenation catalyst is supported by a support material.

8. The method according to claim 1, wherein one, two, or more of the following additional steps are carried out after step (iii):
removing solvent used in step (iii) by evaporation,
chemically converting one, two, or more of said product compounds selected from the group consisting of aliphatic polyols having three or more hydroxy groups and a total number of carbon atoms in the range of from 5 to 6 present in the product resulting from step (iii) to give a product (b) comprising one or more reaction products of said one, two, or more compounds selected from the group consisting of aliphatic polyols having three or more hydroxy groups and a total number of carbon atoms in the range of from 5 to 6,
and
adding one or more additional chemical substances to one, two, or more of said product compounds present in the product resulting from step (iii) selected from the group consisting of aliphatic polyols having three or more hydroxy groups and a total number of carbon atoms in the range of from 5 to 6 so that a reaction mixture results comprising said one, two, or more product compounds selected from the group consisting of aliphatic polyols having three or more hydroxy groups and a total number of carbon atoms in the range of from 5 to 6.

9. The method according to claim 1, wherein the starting material comprising a starting compound selected from the group consisting of aliphatic tricarboxylic acids, esters thereof, anhydrides thereof, and salts thereof is prepared or isolated from plant material.

10. The method according to claim 1, wherein the product comprises
(a) one, two, or more product compounds selected from the group consisting of aliphatic polyols having three or more hydroxy groups and a total number of carbon atoms in the range of from 5 to 6,
wherein said product is selected from the group consisting of
formulations comprising one or more materials selected from the group consisting of peptides, proteins, enzymes, microorganisms, DNA, RNA, and viruses,
paint formulations and adhesive formulations,
reaction mixtures for converting one or more of said aliphatic polyols into polyalkoxylates, wherein the reaction mixture is prepared after step (iii),
reaction mixtures for preparing polymers, wherein the reaction mixture is prepared after step (iii), and
reaction mixtures for preparing esters, acyclic ethers, or cyclic ethers of said one, two, or more product compounds selected from the group consisting of aliphatic polyols having three or more hydroxy groups and a total number of carbon atoms in the range of from 5 to 6, wherein the reaction mixture is prepared after step (iii);
or
(b) one or more reaction products of said one, two, or more product compounds selected from the group consisting of aliphatic polyols having three or more hydroxy groups and a total number of carbon atoms in the range of from 5 to 6,
wherein said product is selected from the group consisting of
products comprising one or more polyalkoxylates, wherein at least one of said polyalkoxylates is prepared from said one, two, or more product compounds selected from the group consisting of aliphatic polyols having three or more hydroxy groups and a total number of carbon atoms in the range of from 5 to 6, in an additional step conducted after step (iii), and
products comprising one or more polymers, wherein at least one of said polymers is prepared from said one, two, or more product compounds selected from the group consisting of aliphatic polyols having three or more hydroxy groups and a total number of carbon atoms in the range of from 5 to 6, in an additional step conducted after step (iii).

11. A product selected from the group consisting of
formulations comprising one or more materials selected from the group consisting of enzymes,
wherein the product comprises one, two, or more product compounds selected from the group consisting of 3-(hydroxymethyl)pentane-1,3,5-triol, propane-1,2,3-trimethanol, 3-(hydroxymethyl)-2-pentene-1,5-diol and 1,3,5-pentanetriol.

12. A method of using a compound selected from the group consisting of aliphatic tricarboxylic acids having a total number of 6 carbon atoms, esters thereof, anhydrides thereof, and salts thereof, the method comprising using the compound as a starting compound in a method according to claim 1 for making
one or more product compounds selected from the group consisting of
aliphatic polyols having three or four hydroxy groups and a total number of carbon atoms of 6,
and
aliphatic polyols having three hydroxy groups and a total number of carbon atoms of 5
or
mixtures of such product compounds.

13. A method of using a heterogeneous hydrogenation catalyst
comprising one or more metals selected from the group consisting of Mn, Re, Fe, Ru, and Os in a total amount of 90 wt.-% or more, based on the total amount of the heterogeneous hydrogenation catalyst,
or
comprising one or more metals selected from the group consisting of Mn, Re, Fe, Ru, and Os in a total amount of 10 wt.-% or more, based on the total amount of the heterogeneous hydrogenation catalyst, in combination with one or more metals selected from the group consisting of Co, Rh, Ir, Ni, Pd, Pt, Cu, Ag, and Au, wherein the total amount of metals selected from the group consisting of Mn, Re, Fe, Ru, Os, Co, Rh, Ir, Ni, Pd, Pt, Cu, Ag, and Au is 98 wt.-% or more, based on the total amount of the heterogeneous hydrogenation catalyst,
in a method according to claim 1.

14. A method of using one, two, or more product compounds selected from the group consisting of aliphatic polyols having three or more hydroxy groups and a total number of carbon atoms in the range of from 5 to 6, the method comprising using the one, two, or more product compounds in a product selected from the group consisting of
formulations comprising one or more materials selected from the group consisting of peptides, proteins, enzymes, microorganisms, DNA, RNA, and viruses,
paint formulations and adhesive formulations,
reaction mixtures for converting one, two or more of said aliphatic polyols into polyalkoxylates,
reaction mixtures for preparing polymers, and
reaction mixtures for preparing esters, acyclic ethers, or cyclic ethers of said one, two, or more product compounds selected from the group consisting of aliphatic polyols having three or more hydroxy groups and a total number of carbon atoms in the range of from 5 to 6.

15. A method of using one, two, or more product compounds selected from the group consisting of 3-(hydroxymethyl)pentane-1,3,5-triol, propane-1,2,3-trimethanol, 3-(hydroxymethyl)-2-pentene-1,5-diol, and 1,3,5-pentanetriol, the method comprising using the one, two, or more product compounds in a product selected from the group consisting of formulations comprising one or more materials selected from the group consisting of enzymes.

16. The method according to claim 2, wherein the starting compound is selected from
the group consisting of alpha-hydroxy aliphatic tricarboxylic acids, esters thereof, anhydrides thereof, and salts thereof,
or
the group consisting of aconitic acid, esters thereof, anhydrides thereof, and salts thereof.

17. The method according to claim 3,
wherein the product resulting in step (iii) comprises one, two, or more product compounds selected from the group consisting of
3-(hydroxymethyl)pentane-1,3,5-triol, propane-1,2,3-trimethanol, and 3-(hydroxymethyl)-2-pentene-1,5-diol,
and
1,3,5-pentanetriol.

18. The method according to claim 4, wherein the solvent provided or prepared in step (ii) is selected from the group consisting of
water,
and
aqueous mixtures comprising water in an amount of more than 50 wt.-%, based on the total amount of the solvent.

19. The method according to claim 6, wherein the heterogeneous hydrogenation catalyst comprises one or more metals selected from the group consisting of Mn, Re, Fe, Ru, and Os in a total amount of 95 wt.-% or more, based on the weight of the total amount of the heterogeneous hydrogenation catalyst.

20. The method according to claim 7, wherein the support material is selected from the group consisting of metal oxides, zeolites, and carbon-based materials.
